(19) United States
(12) Patent Application Publication
(10) Pub. No.: US 2018/0193055 A1
(43) Pub. Date: Jul. 12, 2018

(54) ROTARY DRIVE ULTRASONIC SURGICAL DEVICE AND METHODS OF USE

(71) Applicants: Zhao ying Zhou, Beijing (CN); Yu li Zhang, Beijing (CN); Xiao ning Luo, Beijing (CN); Qing Zhou, Beijing (CN); Wei Tian, Beijing (CN)

(72) Inventors: Zhao ying Zhou, Beijing (CN); Yu li Zhang, Beijing (CN); Xiao ning Luo, Beijing (CN); Qing Zhou, Beijing (CN); Wei Tian, Beijing (CN)

(21) Appl. No.: 15/693,281

(22) Filed: Aug. 31, 2017

(30) Foreign Application Priority Data
Aug. 31, 2016 (CN) 201621033874.8
Aug. 31, 2016 (CN) 201621034195.2

(51) Int. Cl. A61B 17/32; A61B 17/3207; A61B 17/14

(52) U.S. Cl. CPC A61B 17/320068; A61B 2017/320082; A61B 17/142; A61B 17/320758

(57) ABSTRACT

Ultrasonic surgical device is provided. The device includes a shell which holds an energy converter and an amplitude-change pole. The energy converter may include piezoceramic stack that converts electrical signals into mechanical ultrasonic movement in the longitudinal direction of the device. The amplitude-change pole is a mechanical amplifier that uses mass differentials to amplify the ultrasonic motion. A cutting instrument interfaces with the amplitude-change pole. The system also includes a rotary drive device comprising a drive motor also axially arranged with the cutting instrument. The motor rotates the amplitude-change pole, energy converter, and ultimately the cutting instrument. In some embodiments the rate of rotation is at 40-1000 rpm, at 50-650 rpm and at 60-350 rpm depending on configuration. In some cases the user may be enabled to alter the rotational speed within these ranges

### CROSS REFERENCE TO RELATED APPLICATION

This application claims the benefit of Chinese application entitled “The ultrasonic handle and the ultrasonic surgical system, which are used in the ultrasonic surgical system” Chinese application Ser. No. 201621034195.2, filed in the SIPO on Aug. 31, 2016, all of which is incorporated herein by reference.

This application also claims the benefit of Chinese application entitled “The ultrasonic handle and the ultrasonic surgical system, which are used in the ultrasonic surgical system” Chinese application Ser. No. 201621033874.8, filed in the SIPO on Aug. 31, 2016, all of which is incorporated herein by reference.

The present invention is related to commonly owned U.S. application Ser. No. 15/693,246 filed on Aug. 31, 2017 in the USPTO, entitled “Oscillating Drive Ultrasonic Surgical Device and Methods of Use,” the contents of which are incorporated herein by reference.

### BACKGROUND

The present invention relates generally to surgical systems, and specifically to rotary driven ultrasonic surgical devices for cutting bone.

During many surgical situations it may become necessary to cut through bone structures in order to replace faulty orthopedic elements or to access shielded anatomy of the patient. Historically, manual saws have been utilized for these bone cutting processes. More recently, motorized reciprocating saws have been utilized. While saws are effective at cutting through the bone, the saw can result in splintering or rough bone edging which may complicate healing times. When fine tooth saws are utilized, the risk of splintering decreases, and the edges may be made smoother, however significant heat can be generated during the cutting process. Further, regardless of the saw type and tooth spacing, all saws have sharp edges that have the potential to injure surrounding tissue.

In addition to saws, scissoring style cutters may be utilized, such as costotomes, may be employed. These cutters also have the potential to splinter the bone, and may induce crushing damage if not kept sharp and in good condition. Further, these cutters are often limited to use in particular regions of the body or on bone structures of particular diameter.

Recently, sonic cutters have been utilized for bone cutting. Ultrasonic cutters feature precision cutting, higher safety, tissue selectivity and low-temperature operation in a way to increase the ease of use for a surgeon, improve the quality of the surgical operation and reduce/relieve pain in the patients. However, traditional ultrasonic bone cutters are limited in operation by only allowing back-and-forth vibration in a vertical orientation which is not efficient when cutting bone structures. Due to the low efficiency, friction between the incision/wound site is exacerbated resulting in excessive damage to the surrounding tissue. Further, the temperature of cutting surface is raised, which may further compound the problem by resulting in thermal injury of nerves and blood vessels around the wound.

It is therefore apparent that an urgent need exists for improved ultrasonic bone cutting devices that effectively cut the bone tissue, minimize adjacent wound damage, and are easily employed by a surgeon.

### SUMMARY

To achieve the foregoing and in accordance with the present invention, a rotary drive ultrasonic surgical device is provided.

In some embodiments, the ultrasonic surgical device includes a shell which holds an energy converter and an amplitude-change pole. The energy converter may include piezoceramic stack that converts electrical signals into mechanical ultrasonic movement in the longitudinal direction of the device. The amplitude-change pole is a mechanical amplifier that uses mass differentials to amplify the ultrasonic motion. A cutting instrument interfaces with the amplitude-change pole.

The system also includes a rotary drive device comprising a drive motor also axially arranged with the cutting instrument. The motor rotates the amplitude-change pole, energy converter, and ultimately the cutting instrument. In some embodiments the rate of rotation is at 40-1000 rpm, at 50-650 rpm and at 60-350 rpm depending on configuration. In some cases the user may be enabled to alter the rotational speed within these ranges.

The motor may be connected to a deceleration mechanism comprising a planetary gear array. This may include a planet carrier, sun wheel and a gear ring. The carrier may be fixed to the inside surface of the shell.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawing, in which:

FIG. 1 shows a diagram of a rotary drive ultrasonic surgical device.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

Further, terminologies, such as “Installation”, “Linkage”, “Connection”, “Fixation”, should be understood in a broad sense unless there exists addition specifications and limitations. For example, it could be permanent connection, detachable connection or all-in-one, electric connection or intercommunication, direct connection or indirect connection through intermediation, it could also be the inner connection or the interconnection of the two components. As to the ordinary technical personnel who could analyze the implications of the above terminologies in the utility model according to the specific circumstances.

The present invention relates to an ultrasonic surgical device. As noted previously, current ultrasonic systems operate by vibrating the cutting instrument in a vertical orientation in a back-and-forth manner. This cutting motion causes low-efficiencies when cutting through bone structures. Low efficiency cuts have a number of drawbacks: they increase friction at the wound site, and result in increased temperature at the point of cutting. The friction on the incision site may result in direct damage to surrounding tissue. Damage may cause later complications, longer healing time, and greater post-operative pain for the patient. The added heat at the cutting location may force the surgeon to cut for shorter periods of time to avoid undue thermal damage to the surrounding tissue. This may increase the difficulty of use for the surgeon. Further even when taking frequent breaks to allow the cut location and tool to cool, small vessels and nerves proximate to the cutting location will be damaged by the excess heat regardless.

Lastly, the inefficient cutting of these traditional systems simply prolongs the cutting process. For larger bone structures this may cause fatigue for the surgeon. Further, the longer a patient remains in surgery, generally the lower the expected outcome, and increased surgical cost. As such, there is incentive to shorten surgical procedures.

The presently described ultrasonic surgical device improves on these older systems by allowing rotation of the cutting instrument in addition to the ultrasonic vibration. This rotation enables more efficient cutting of bone structures, thereby reducing friction on surrounding tissues, reduced heat generation and shorter overall cutting times. The present system also addresses drawbacks related to osteotomy grinding and fatigue caused to the surgeon due to the strong vibrational forces caused by traditional ultrasonic cutters.

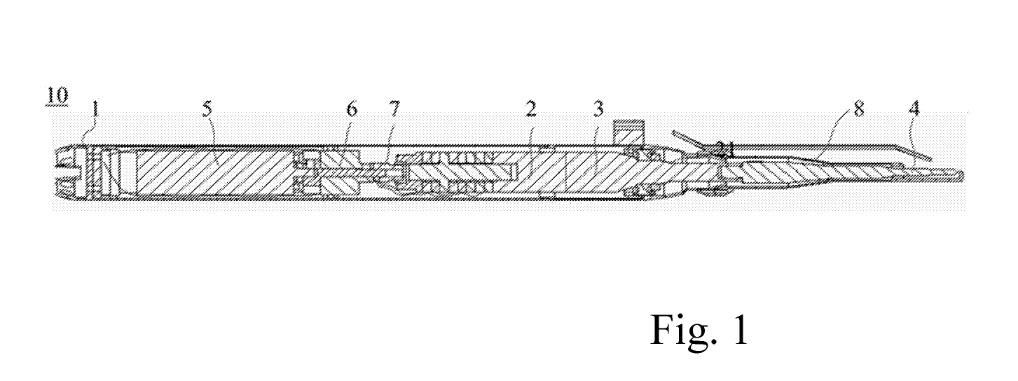

To facilitate discussion, attention is drawn to FIG. 1, which provides a diagram of the ultrasonic surgical device handle, as seen generally at 10. In this example illustration, the ultrasonic device handle is seen as comprising an outer shell 1, an energy converter 2, amplitude-change pole 3, an ultrasonic bone cutter 4, a rotary drive motor 5, a conductive slip ring 6, a coupler 7, and a protective cover 8.

The energy converter 2, is set in the shell, which is used to convert the ultrasonic signal into a mechanical wave. As with many traditional ultrasonic devices, the energy converter 1 may include a piezoceramic stack that converts the electrical signals into the ultrasonic vibrations.

Similarly, the amplitude-change pole 3 is set in the shell. Specifically, the amplitude-change pole 3 is connected between the ultrasonic bone cutter 4 and energy converter 2. The amplitude-change pole 3 in some embodiments may include a mechanical amplifier that adjusts the amplitude of the cutting instrument based on the configuration of masses on each end of the structure. The amplitude-change pole 3 magnifies the amplitude of the mechanical wave of energy converter 2, and then delivers the mechanical wave to the ultrasonic bone cutter 4 in a manner that causes the ultrasonic bone cutter to vibrate in a vertical direction. The term “vertical” in this context is in relation to the longitudinal axial orientation of the cutting device.

The rotary drive device 5 includes a drive motor. The drive motor is able to cause the ultrasonic bone cutter 4, amplitude-change pole 3 and energy converter 2 to rotate around the central axis of the ultrasonic bone cutter 4, with a rotational speed between 40 rpm to 1000 rpm, in a specific example. In some other systems, the rate of rotation may be between 50-650 rpm, 60-350 rpm and 40-400 rpm.

The drive motor 5 may be installed in the back-end of shell 1. The motor shaft of drive motor 5 may drive the rotation of energy converter 2 and amplitude-change pole 3 through the connection between the coupler 7 and the energy converter 2 and amplitude-change pole 3 to create the circumstantial rotation of the ultrasonic bone cutter 4 which is installed in the front-end of amplitude-change pole 3. This rotational motion is compounded with the longitudinal vibration generated by the energy converter 2.

The ultrasonic bone cutter 4 having this compound motion with longitudinal vibration and circumferential rotation facilitates the incision, drill and grinding of the bone, which not only simplifies the surgical process through enhancing the cutting efficiency, but also reduce the friction between ultrasonic bone cutter 4 and wound surface in a way to decrease the cutting temperature of the wound surface and avoid the break due to the stress concentration of ultrasonic bone cutter 4. At the same time, the operator is able to hold the ultrasonic handle 10 conveniently and obtain the improved efficiency when cutting and grinding the bone through controlling the rotating speed of drive motor 5 within 40 rpm to 1000 rpm and controlling the amplitude of vibration of the ultrasonic handle 10. The rotating speed mentioned above can be configured by moderating the twisting force delivered by the motor to the ultrasonic bone cutter. By varying the speed of rotation, a number of problems can be addressed, such as the problem of inefficiency in cutting due to the low-speed rotation and the problem of being unable to grind rigid bone due to too high a rate of rotation. The ultrasonic bone cutter is thus able to obtain the perfect required balance between grinding and cutting by allowing the rotation speed to be modified.

The ultrasonic handle 10 may be used in a larger ultrasonic surgical system by coupling it to an energy supply, and a display system that allows configuration of the rotational speeds and ultrasonic amplitude. A larger ultrasonic surgical system may include, in addition to the dedicated ultrasonic bone cutter, additional instruments to allow for incision, drilling and grinding in the bone structure.

The conductive slip ring 6 may be set between drive motor 5 and energy converter, which enables transmission of the electronic ultrasonic signal to the energy converter 2. The protective cover 8 may be set in the outside of the ultrasonic bone cutter 4, in order to protect the ultrasonic bone cutter 4, thereby avoiding the intrusion of particles, debris and biological materials from entering the system.

In some example, the rotating speed of drive motor is from 50 rpm to 650 rpm, which may further improve the twisting force transmitted from the motor to the ultrasonic bone cutter 4 so as to enhance the efficiency in cutting and grinding of the ultrasonic bone cutter 4. In another embodiment, rotational speed of the drive motor 5 is from 60 rpm to 350 rpm, which could represent a twisting force transmitted from motor to the ultrasonic bone cutter in a way to enhance the efficiency in cutting and grinding of the ultrasonic bone cutter 4.

The dive motor 5, energy converter 2, bone cutter 4 and amplitude-change pole 3 are coaxially arranged. By arranging these components on axis, the radial dimension of the handle 10 may be reduced in size, thereby making the device fit the hand of the surgeon in a more convenient and ergonomically proper manner.

The output driveshaft of drive motor 5 may engage a decelerating mechanism (not illustrated). In some embodiments, the decelerating mechanism may be comprised of a planetary gear. The twisting force delivered from the motor to the ultrasonic bone cutter 4 may be improved by the decelerating mechanism to ensure that starts and stops are smooth and gradual, thereby limiting jerks or sudden rotational shifts, which in turn enhance the effect of cutting and grinding on the bone.

In some embodiments, the decelerating mechanism with the planetary gear includes a sun wheel, a planet carrier and a gear ring. The sun wheel is used to power an input end that is connected with the drive motor 5. The gear ring constitutes the power output end, and the planet carrier is fixed. Specifically, the planet carrier is fixed in the inner wall of the shell 1. The sun wheel and ultrasonic bone cutter 4 are arranged coaxially. The decelerating mechanism with the planetary gear may reduce the speed of the bone cutting instrument while increasing torque through gearing ratios. The larger torque facilitates the rotation of the ultrasonic bone cutter, which typically cannot be supplied without larger sized motor elements. As such, the decelerating mechanism with the planetary gear itself is a compact structure, and allows a smaller motor unit to rotate the cutting instrument with the required level of torque. In addition does the larger torque provide better unit operation, it further enables the device to operate in a more robust and reliable manner.

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. An ultrasonic surgical device comprising:
a shell having a central axis;
an energy converter contained within the shell;
an ultrasonic bone cutting instrument;
an amplitude-change pole contained within the shell and located between the ultrasonic bone cutting instrument and the energy converter; and
a rotary drive device comprising a drive motor, wherein the drive motor causes rotation of the central axis at a rate of rotation between 40 to 1000 rotations per minute (rpm).

2. The ultrasonic surgical device of claim 1, wherein the rate of rotation is between 50-650 rpm.

3. The ultrasonic surgical device of claim 1, the rate of rotation is between 60-350 rpm.

4. The ultrasonic surgical device of claim 1, wherein the bone cutting instrument, the energy converter, the amplitude-change pole, and the drive motor are coaxially arranged.

5. The ultrasonic surgical device of claim 1, further comprising a deceleration mechanism coupled to the drive motor.

6. The ultrasonic surgical device of claim 5, wherein the deceleration mechanism comprises a planetary gear array.

7. The ultrasonic surgical device of claim 5, wherein the planetary gear array includes a sun wheel, a planet carrier and a gear ring, wherein the sun wheel is coupled with the drive motor, wherein the gear ring constitutes the power output end, and wherein the planet carrier is fixed.

8. The ultrasonic surgical device of claim 7, wherein the planet carrier is fixed to an inner wall of the shell.

9. The ultrasonic surgical device of claim 7, wherein the sun wheel and bone cutting instrument are coaxially arranged.

10. An ultrasonic surgical system comprising:
an ultrasonic surgical device as presented in claim 1;
a power supply; and
a display system configured to display the rate of rotation.

11. A method of treating target tissue within a body using an ultrasonic surgical device according to claim 1, wherein the method includes rotating an ultrasonic bone cutting instrument and physically contacting the ultrasonic bone cutting instrument the target tissue, by an operator, to induce cutting, wherein an rate of rotation for the ultrasonic surgical device is configured by the operator of the device.

12. The method of claim 11, wherein the target tissue is a bone structure.
